(19) United States
(12) Patent Application Publication
(10) Pub. No.: US 2020/0397315 A1
(43) Pub. Date: Dec. 24, 2020

(54) WEARABLE FLUIDIC DEVICE AND SYSTEM WITH INTEGRATED ELECTRONICS

(71) Applicant: Epicore Biosystems, Inc., Cambridge, MA (US)

(72) Inventors: Milan S. Raj, Natick, MA (US); Stephen P. Lee, Ann Arbor, MI (US); Alexander J. Aranyosi, Medford, MA (US); Jeffrey B. Model, Cambridge, MA (US); Roozbeh Ghaffari, Cambridge, MA (US)

(73) Assignee: Epicore Biosystems, Inc., Cambridge, MA (US)

(21) Appl. No.: 16/906,649

(22) Filed: Jun. 19, 2020

Related U.S. Application Data
(60) Provisional application No. 62/863,595, filed on Jun. 19, 2019.

(51) Int. Cl. A61B 5/0205; A61B 5/145; A61B 5/00; A61B 5/0285; A61B 5/11; A61B 5/0488; A61B 5/053; A61B 5/1455

(52) U.S. Cl. CPC A61B 5/02055; A61B 5/02405; A61B 5/0022; A61B 5/0285; A61B 5/112; A61B 5/1116; A61B 5/0488; A61B 5/053; A61B 5/6801; A61B 5/14546; A61B 5/4845; A61B 5/14532; A61B 5/486; A61B 5/1455; A61B 5/14517

(57) ABSTRACT

A system for collecting and analyzing sweat includes a sweat sensing device having at least one sensor configured to sense one of physiologic information, biologic information, biochemical information, and biometric information, from skin of a person to whom the sweat sensing device is attached. A wireless transmitter is mounted to the sweat sensing device and electrically connected to the at least one sensor. An interactive console is configured to produce a real time feedback to an operator of the system for collecting and analyzing sweat, includes a wireless receiver, and is in wireless communication with the sweat sensing device. The interactive console is further configured to receive data from the at least one sensor, display the data received from the at least one sensor, adjust an environmental condition based on the sensor data received, and display the adjustment to the environmental condition.

### BACKGROUND OF THE INVENTION

This invention relates in general to an epidermal system for collecting, measuring, and/or monitoring sweat rate, sweat loss, sweat composition, biochemical information, and/or biologic information about one or more persons. In particular, this invention relates to an improved epidermal system or electronics-enabled microfluidics sensor (EEMS) system that includes a disposable, wearable, sweat sensing device with integrated biosensors and microfluidics that captures or collects a skin bio-fluid, such as sweat, and measures or monitors sweat rate, sweat loss, sweat, sweat composition, and biochemical and/or biologic information, and transmits this information to a remote processor.

Cardiovascular disease is a leading cause of mortality in the United States and accounts for about 30% of all global deaths. Millions of adults in the U.S. live with hypertension and congestive heart failure (CHF). Significantly, there is a broad range of adverse effects and co-morbidities associated with hypertension and CHF, including stroke, angina, and myocardial infarctions, which burden the health care system at a cost of about $30 billion US per year. With recent advances in wearable biosensor technologies, there is potential to measure cardiovascular health, electrolyte balance, blood pressure, and hemodynamics metrics conveniently, unobtrusively, and without bulky equipment.

The possibility of traumatic brain injury (TBI) is a concern for those participating in high-impact athletic activities, for example, American football, soccer, and ice hockey, and may lead to severe, neuro-degenerative diseases, also known as dysautonomia, whose symptoms may present across a number of different biomarkers. In particular, a longitudinal view of elevated core body temperature, heart and respiratory rates, blood pressure, and sweat rate may indicate damage to the autonomic nervous system (ANS) and provide diagnostic evidence of dysautonomia. Key to reducing morbidity in this subclass of TBI patients is the ability to properly identify an abnormal change in these biomarkers concurrently and over an extended period of time in an unobtrusive and efficient manner.

Existing wearable devices are primarily based on electronic sensing of inertial and/or electrical and optical signals. These systems rely on close coupling to the human body, but at the same time require protection from the unique hazards introduced by such coupling, including deformation and exposure to sweat and other liquids. The integration of electronic and microfluidics-based biochemical sensors is of keen interest to clinicians, researchers, and others focused on human performance. However, current manufacturing processes do not support such tight integration of the required components.

Cardiac monitoring solutions based on non-invasive monitoring devices, for example Holter monitors, blood pressure cuffs, event monitors, implantable devices such as pacemakers and implantable pressure sensors, and fluid retention/dehydration monitoring systems such as body-mass scales and bio-impedance measurement systems, have dramatically improved diagnostic yield and CHF/hypertension patient outcomes. Although the utility of existing diagnostic technologies is well established, there are inherent tradeoffs between the physical packaging, the suite of sensing modalities, data quality, usability, comfort, and cost of existing solutions. Moreover, conventional monitoring devices are largely incompatible with the fragile skin of elderly patient populations. There is growing interest to monitor key vital signs in combination with entirely new classes of soft/flexible wearable biosensors that are imperceptible to the user, low cost, portable, and wirelessly connected for deployment in the workplace and in the home. Patient adoption is increasingly driven by these factors in conjunction with ownership of smart phones. It is estimated that about 64% of American adults own a smart phone, with an estimated 1.5 billion users globally. These devices serve as communication portals, facilitating wireless connectivity across numerous ancillary cardiac monitoring devices, which in turn, generate an expansive body of clinical data.

Holter monitors, event monitors, and loop recorders represent the clinical standard of care in cardiology, and have been used to assess CHF patients during the post-discharge period. Hypertension, a precursor to CHF, is associated with increased extracellular sodium, which in turn affects kidney function leading to increased water retention and decreased sodium excretion. However, there is currently no solution that monitors cardiac function, fluid retention, and electrolyte balance in a single wireless and wearable design.

A novel, non-disruptive monitoring solution that integrates flexible-hybrid electronics (FHE) and biochemical sensing to measure key biometrics could fundamentally change the way CHF rehabilitation and TBI management are completed during a post-discharge period when patients are the most vulnerable and susceptible to readmission. The manufacture of such integrated systems has broad application beyond CHF, in athletic performance, military training, and overall wellness.

There is growing interest to monitor key vital signs in combination with entirely new classes of soft/flexible wearable biosensors that are imperceptible to the user, low cost, portable, and wirelessly connected for deployment in the workplace and in the home.

Thus, it would be desirable to provide an improved, wearable EEMS system that includes a disposable, wearable, sweat sensing device with integrated biosensors and microfluidics that is soft and flexible, imperceptible to the user, low cost, portable, wirelessly connected for deployment in the workplace and in the home, captures or collects a skin bio-fluid, such as sweat, measures or monitors sweat rate, sweat loss, sweat, sweat composition, and biochemical and/or biologic information, and transmits this information to a remote processor.

### SUMMARY OF THE INVENTION

This invention relates to a wearable EEMS system that includes a disposable, wearable, sweat sensing device with integrated biosensors and microfluidics that is soft and flexible, imperceptible to the user, low cost, portable, wirelessly connected for deployment in the workplace and in the home, captures or collects a skin bio-fluid, such as sweat, measures or monitors sweat rate, sweat loss, sweat, sweat composition, and biochemical and/or biologic information, and transmits this information to a remote processor.

A system for collecting and analyzing sweat includes a sweat sensing device having at least one sensor configured to sense one of physiologic information, biologic information, biochemical information, and biometric information, from skin of a person to whom the sweat sensing device is attached. A wireless transmitter is mounted to the sweat sensing device and electrically connected to the at least one sensor. An interactive console is configured to produce a real time feedback to an operator of the system for collecting and analyzing sweat, includes a wireless receiver, and is in wireless communication with the sweat sensing device. The interactive console is further configured to receive data from the at least one sensor, display the data received from the at least one sensor, adjust an environmental condition based on the sensor data received, and display the adjustment to the environmental condition.

Various aspects of this invention will become apparent to those skilled in the art from the following detailed description of the preferred embodiment, when read in light of the accompanying drawings.

### BRIEF DESCRIPTION OF THE DRAWINGS

FIG. 1 is a block diagram of one embodiment of a wearable EEMS system according to this invention showing multiple sensors and sweat micro-fluidic analysis components.

FIG. 2A is a schematic illustration of a first embodiment of a method for optical measurement of sweat volume and concentration for use in the wearable EEMS system of FIG. 1 and using the attenuation of light as it propagates through a sweat sample in a transmissive mode.

FIG. 2B is a schematic illustration of a second embodiment of a method for optical measurement of sweat volume and concentration for use in the wearable EEMS system of FIG. 1 and using the attenuation of light as it propagates through a sweat sample in a reflectance mode.

FIG. 3A is a schematic illustration of a method for sweat volume measurement using a superabsorbent polymer (SAP) and metal plates shown prior to sweat being combined with the SAP.

FIG. 3B is a schematic illustration of a method for sweat volume measurement using a SAP and metal plates shown in FIG. 3A after sweat is combined with the SAP.

FIG. 4A is a schematic illustration of one embodiment of a portion of the wearable EEMS system illustrated in FIG. 1 showing one sweat collection channel with an analyte and one sweat collection channel without an analyte.

FIG. 4B is a schematic illustration of one embodiment of a conductive measurement circuit used to measure conductance in FIG. 4A.

FIG. 5A is a diagram illustrating a first embodiment of a method of measuring impedance of a sweat channel in the wearable EEMS system illustrated in FIG. 1 showing amplifier output being directed to a diode detector circuit.

FIG. 5B is a diagram illustrating a second embodiment of a method of measuring impedance of a sweat channel in the wearable EEMS system illustrated in FIG. 1 showing amplifier output being directed to an anti-aliasing filter.

FIG. 6 is a schematic illustration of one embodiment of a portion of the wearable EEMS system illustrated in FIG. 1 showing sweat propagation through an inlet and pushing an air bubble that then urges a volume of an APA on to the skin of a user.

FIG. 7 is a perspective view of a one embodiment of an improved sweat collection device with integrated biosensors and microfluidics for use with the wearable EEMS system illustrated in FIG. 1.

FIG. 8 is a schematic, cross-sectional view of the improved sweat collection device with integrated biosensors and microfluidics illustrated in FIG. 7.

FIG. 9 is a diagram showing one embodiment of the placement of the improved sweat collection device with integrated biosensors and microfluidics on a user and showing the user also wearing a smart watch.

FIG. 10A is perspective view of the improved sweat collection device illustrated in FIG. 9 showing an outwardly facing surface thereof.

FIG. 10B is perspective view of the improved sweat collection device illustrated in FIGS. 9 and 10A showing an inwardly facing surface thereof.

FIG. 11 is a graph illustrating one example of a comparison of a photoplethysmography (PPG) waveform taken from a user's forearm and a PPG waveform taken from the user's wrist.

FIG. 12 is a perspective view of a conventional sweat collection device.

FIG. 13 is a cross-sectional view taken along the line 13-13 of FIG. 12.

FIG. 14 is an enlarged cross-sectional view of the portion of the sweat collection device within circle 14 of FIG. 13.

FIG. 15 is a plan view of the conventional sweat collection device illustrated in FIGS. 12 through 14.

FIG. 16 is a cross-sectional view taken along the line 16-16 of FIG. 15.

FIG. 17 is an enlarged cross-sectional view of the portion of the sweat collection device within circle 17 of FIG. 16.

FIG. 18 is an exploded perspective view of the sweat collection device illustrated in FIGS. 14 through 16.

### DETAILED DESCRIPTION OF THE PREFERRED EMBODIMENT

The present invention is directed to a wearable electronics-enabled microfluidics sensor (EEMS) system for digital health applications that rely on the measurement of various sweat biomarkers, sweat rate, and physiological signals from the human body. The wearable EEMS system includes a disposable, wearable, sweat sensing device with integrated biosensors and microfluidics that is soft and flexible, imperceptible to the user, low cost, portable, wirelessly connected for deployment in the workplace and in the home, captures or collects a skin bio-fluid, such as sweat, measures or monitors sweat rate, sweat loss, sweat, sweat composition, and biochemical and/or biologic information, and transmits this information to a remote processor.

These data may be analyzed locally on the sweat sensing device, or in aggregate to provide targeted insight and actionable data endpoints without the need for external processing. The sweat sensing device may communicate with a display unit and/or a smart device for data visualization and user interfacing.

The wearable sweat sensing device collects the sweat from persons such as athletes, military personnel, patients, including remote patients, e.g., hyperhidrosis patients, drug rehabilitation patients, and newborns.

As described in detail below, the wearable sweat sensing device may be configured as a disposable, skin-like epidermal device with integrated biosensors and microfluidics that is wireless and imperceptible on the skin of patients. As will be described in detail below, the sweat sensing device of the EEMS system may contain a microfluidics substrate that houses flexible electronics and a network of underlying microfluidic channels and chemical assay wells for measuring sweat rate and performing biochemical analysis of sweat. The microfluidics substrate may be physically coupled to a battery, electrodes, light emitting diode (LED)/optical sensor, temperature sensor, and accelerometer. These sensors enable electrocardiography (ECG), photoplethysmography (PPG), bio-impedance, and motion sensing, and are supported by an onboard microprocessor and wireless connectivity, such as Bluetooth® capable radio or via other wireless communication methods, for algorithmic processing and wireless data transfer.

The EEMS system provides skin-like properties that enable intimate and imperceptible integration on the body of a user in ways that are impossible to achieve with existing semiconductor fabrication technologies. As a result, clinicians will be able to monitor motion, bio-impedance, ECG and/or PPG, and sweat composition in patients remotely for up to one week immediately following discharge from the hospital. The integration of FHE and biochemical sensors into a single manufacturable system has broad clinical applications in obesity, sports, diabetes, and other areas.

In accordance with embodiments described herein, the EEMS system may integrate body-worn sensors that sense sweat loss and sweat rate, as well as physiological information such as heart rate, electromyography (EMG), ECG, electroencephalogram (EEG), PPG, respiration, blood pressure, galvanic skin response, posture, gait, and motion, and send this information to a smartphone, smartwatch, or digital interactive console (not shown) wirelessly. Non-limiting examples of a digital interactive console include an environment controller console, an athlete weighing station console, such as a smart scale console, and a heart monitoring station, each of which may be configured to provide a desired additional level of input to the associated software.

Additionally, the wearable sweat sensing device may include smart sensors that process the physiologic, biochemical markers, and/or biometric information needed to derive other metrics such as the content of lactate, alcohol, glucose, vitamin c, cortisol, or other biomarkers in sweat, emotional state, stress levels, anxiety and fatigue, and send this information to the interactive console. The smartphone and/or the console may use the physiologic and/or biometric information and/or the derived metrics to change the atmosphere, level of difficulty, dialogue, video action, and other aspects of the audio and/or video presentation to customize the sports, gaming, or virtual environment for the user. This information may also be used to provide a safe operating environment by shutting down the system console if one or more of the sensors indicate a potentially unsafe condition of the user, such as their heart rate or respiration exceeding a threshold, or their EEG signals indicating unsafe brain activity, such as may lead to a seizure or a stroke.

In additional embodiments of the invention, multiple people and/or subjects may be monitored by one or more sweat sensing devices that indicate one or more conditions of some or all of the people and/or subjects. The conditions may include physical conditions, such as location and motion of the person or subject during daily activity, sport event and/or during sleep cycles.

In further embodiments of the invention, data collected from multiple wearers and/or external systems may be combined and/or compared to provide actionable information. As a non-limiting example, referees of an American football game may combine sensor data from multiple players with play-clock data to distinguish between false-start and neutral-zone-infraction penalties. As a non-limiting example, coaches or automated systems may compare the rate of change of one or more biometric measures from multiple players to help determine if any players need to be removed from the game for rest, recovery, or medical treatment.

In other embodiments of the invention, the sensed information about one or more persons' or subjects' sweat rate, sweat composition, and physiology may be collected and processed or analyzed and used as a direct input, or used to select or modify an input to an interactive environment control system, including but not limited to a sports training console, and a virtual reality or augmented reality controller, that controls the environment experienced by the person or subject. The virtual environment control system may include an application programming interface (API) that enables direct external inputs to control or influence one or more aspects of the interactive environment. The sensed information may be used by players, coaches, or physicians to provide feedback to patients, or to analyze data across patient populations and geographies.

In additional embodiments of the invention, sweat from the wearer may be used to control and/or modulate the delivery of pharmaceuticals, and/or other chemicals, hereafter referred to as active pharmaceutical agents (APAs), to the skin, bloodstream, or other skin-accessible tissue. The generation of sweat creates a fluid pressure that may be used to drive flow of the APA. In accordance with some embodiments of the invention, a relationship between sweat fluid pressure and the resulting volumetric flow of the APA may be controlled by a valve integrated into the system.

In some embodiments of the invention, the conductance between two electrodes of the FHE circuit is negligible in the absence of sweat and high in the presence of sweat. This difference may serve as a binary switch to indicate the presence of sweat in the system.

In additional embodiments of the invention, the conductance between two electrodes of the FHE circuit increases over time as sweat is collected. Measurements of the conductance as a function of time may then be used to determine the rate of sweat production.

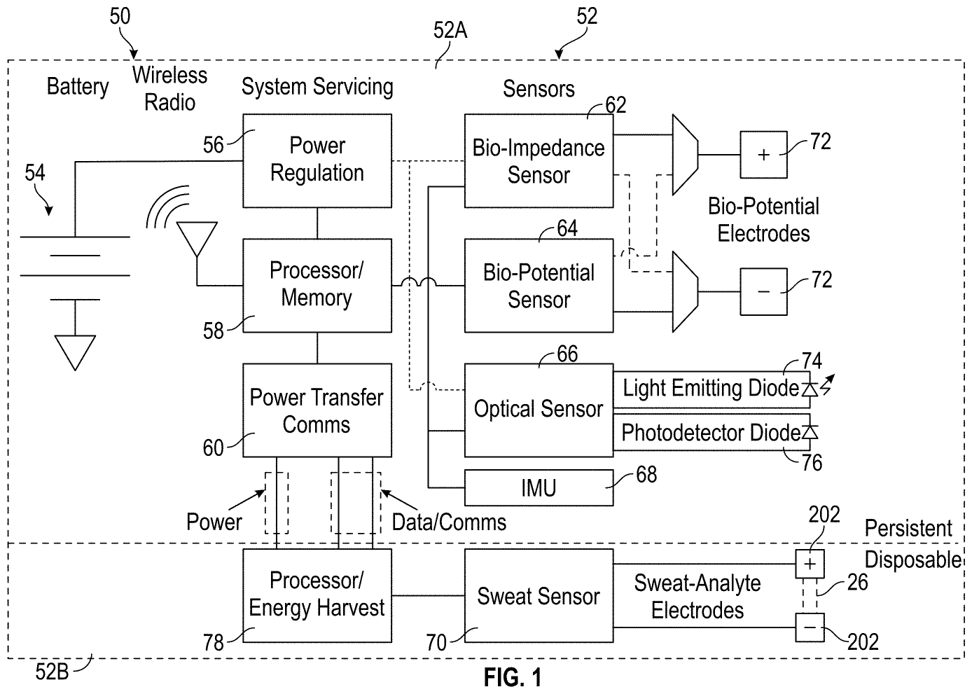

Referring now to the drawings, there is illustrated in FIGS. 1 through 11 an improved, wearable electronics-enabled microfluidics sensor (EEMS) system 50 and various embodiments of components thereof, in accordance with this invention and described in detail below.

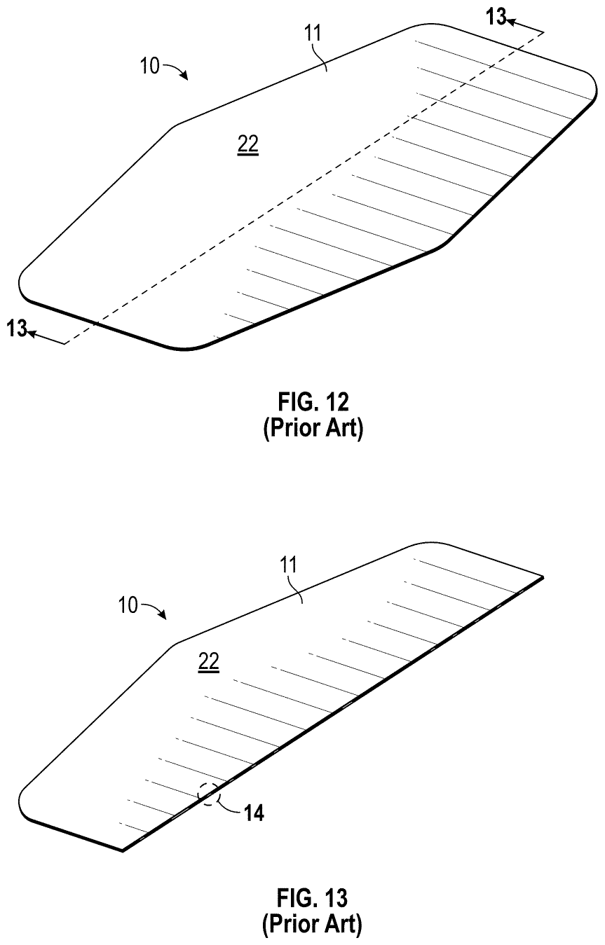

Referring again to the drawings, there are illustrated in FIGS. 12 through 18 examples of an embodiment of a known sweat sensing device 10. The illustrated sweat sensing device 10 is conventional in the art, has been described and illustrated in PCT Application No. PCT/US18/43430, and is intended to illustrate one way that an improved sweat collection device 52 of the wearable EEMS system 50 according to this invention may be constructed.

The sweat sensing device 10 includes a substantially flexible body 11 having a first or upper layer 12, a second layer 14, a third layer 16, a fourth layer 18, and a fifth or lower layer 20. The upper layer 12 has a first or outwardly facing surface 22. The lower layer 20 has a second or skin-facing surface 24. An adhesive is applied to the skin-facing surface 24, and the skin-facing surface 24 is covered by a removable adhesive liner 25 formed from any desired flexible and air/oxygen impermeable material.

The illustrated first layer 12 and the illustrated fifth layer 20 are formed from clear polyurethane having a thickness of about 0.004 inches (0.10 mm) Alternatively, the first layer 12 and the fifth layer 20 may be formed from other desired soft, flexible, and clear material, such as silicone, polyethylene, polyethylene terephthalate (PET), or polyurethane. If desired, the fifth layer 20 may be formed from an opaque material. The first layer 12 and the fifth layer 20 may also have other desired thicknesses. For example, the first layer 12 may have a thickness within about 0.002 in to about 0.006 in (about 0.05 mm to about 0.15 mm), and the fifth layer 20 may have a thickness within about 0.001 in to about 0.004 in (about 0.025 mm to about 0.10 mm)

The illustrated third layer 16 is formed from clear silicone having a thickness of about 0.005 inches (0.127 mm) Alternatively, the third layer 16 may be formed from other desired soft, flexible, and clear material, such as polyurethane, polyester, or PET, and may have other desired thicknesses, such as within about 0.004 in to about 0.006 in (about 0.10 mm to about 0.15 mm)

The illustrated second layer 14 and the illustrated fourth layer 18 are formed from clear acrylic PSA having a thickness of about 0.002 inches (0.50 mm). The second and fourth layers 14 and 18 are adhesive layers that bond the first layer 12, the third layer 16, and the fifth layer 20 together. The material chosen for the adhesive second and fourth layers 14 and 16 may vary based on the material of the layers to which they are applied. For example, a silicon adhesive layer may be chosen to a bond silicon layers together. Alternatively, the second and fourth layers 14 and 18 may have other desired thicknesses, such as within about 0.001 in to about 0.004 in (about 0.025 mm to about 0.10 mm) If desired, the first layer 12, the third layer 16, and the fifth layer 20 may be directly bonded together by any conventional means, such as by ultrasonic welding.

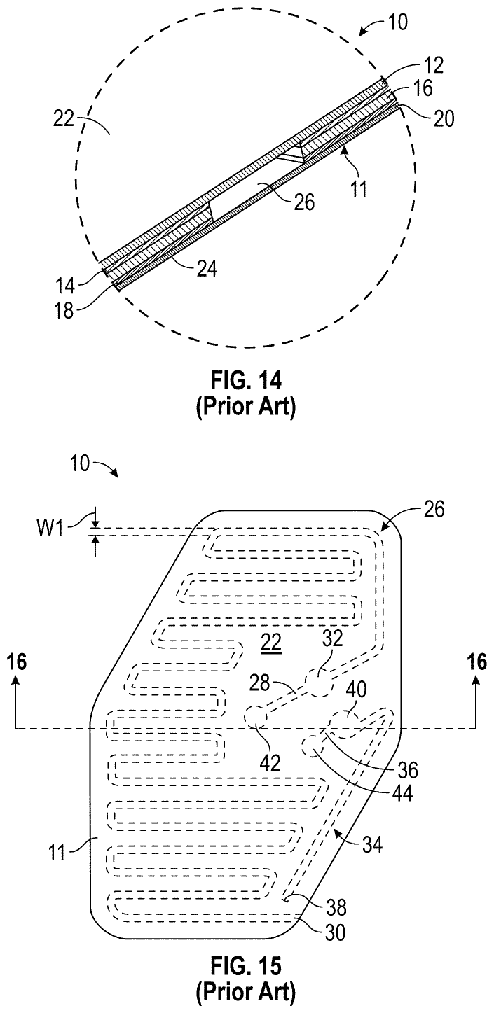

One or more sweat channels may be formed in at least the third layer 16. As shown in embodiment of the sweat sensing device 10 illustrated in FIGS. 14 through 18, a first microfluidic or sweat channel 26 is formed in the third layer 16 and defines a serpentine pathway. Alternately, and as shown in the illustrated embodiment of the sweat sensing device 10, the first sweat channel 26 is also formed in the second and fourth layers 14 and 18, respectively. The first sweat channel 26 has a sweat inlet end 28 and a sweat outlet end 30 at a peripheral edge of the sweat sensing device 10 and positioned to allow sweat to exit the first sweat channel 26. The first sweat channel 26 may also include a biochemical or assay well 32 near the sweat inlet end 28.

Additionally, a sweat channel may be formed such that portions of the sweat channel are variously formed in the second layer 14, the third layer 16, and in the fourth layer 18, or in combinations of layers, such as in the second and third layers 14 and 16 and in the third and fourth layers 16 and 18. Varying the height of the sweat channel throughout its length in this manner allows areas of greater sweat channel height to be positioned in the flexible body 11 as a visual indicator wherein a color change within the portions of the sweat channel having the greater height may be more easily seen because a larger volume of dye therein may appear darker in color.

When a sweat channel is formed having different heights throughout its length, i.e., when portions of the sweat channel are variously formed in the layers thereof, the sweat channel may crossover itself, allowing for a longer sweat channel without the need to increase the size of the sweat sensing device 10.

As best shown in FIG. 15, a second microfluidic or sweat channel 34 is also formed in the second, third, and fourth layers 14, 16, and 18, respectively. The second sweat channel 34 has a sweat inlet end 36 and a second end 38 that, unlike the first sweat channel 26, does not define a sweat outlet. The second sweat channel 34 may also include a biochemical assay well 40 near the sweat inlet end 36.

The lower layer 20 may have fluid or sweat inlet ports in fluid communication with the sweat channels. As best shown in FIG. 15, the lower layer 20 includes a first sweat inlet port 42 in fluid communication with the first sweat channel 26, and a second sweat inlet port 44 in fluid communication with the second sweat channel 34. In the illustrated embodiment of the sweat sensing device 10, the biochemical assay wells 32 and 40 extend through the lower layer 20 to allow for the insertion a chemical assay therein.

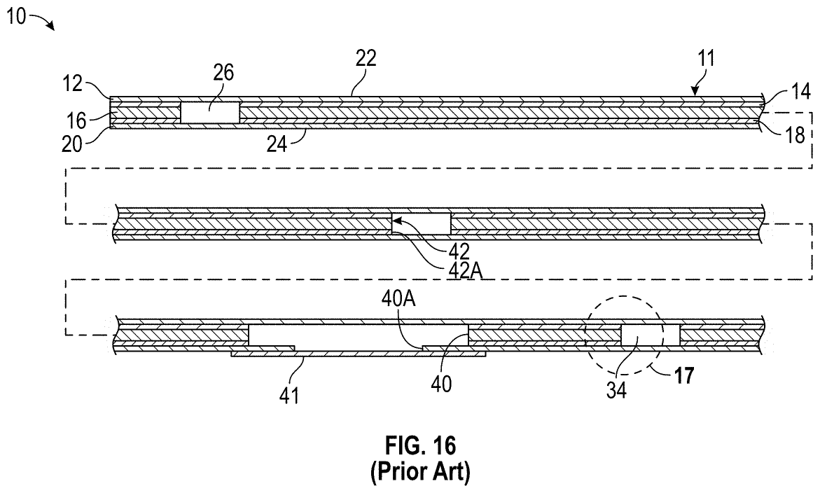

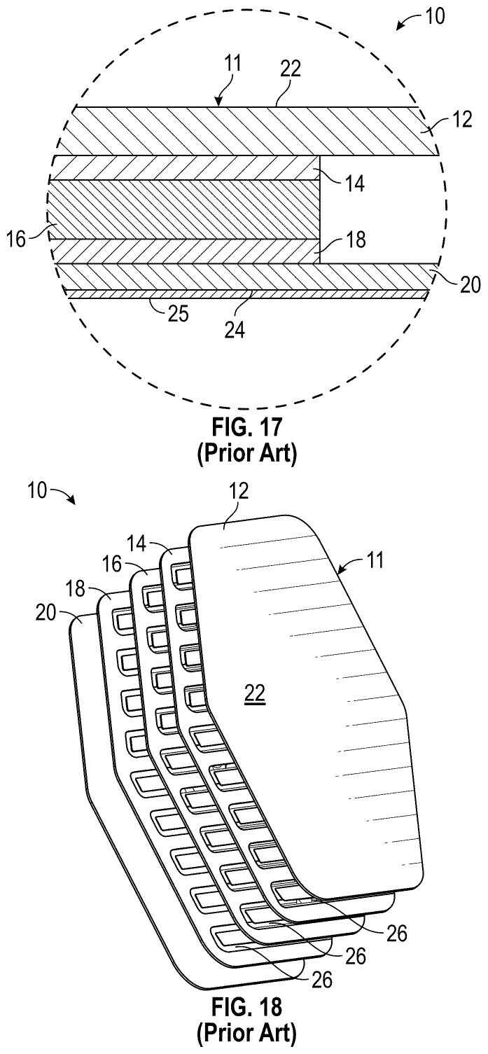

As shown in FIG. 16, a portion 42A of the first sweat inlet port 42 in the lower layer 20 may be smaller than the portions of the first sweat inlet port 42 formed in the second, third, and fourth layers, 14, 16, and 18, respectively. Similarly, a portion 40A of the biochemical assay well 40, and a portion (not shown) of the biochemical assay well 32, in the lower layer 20 may be smaller than the portions of the biochemical assay wells 40 and 32 formed in the second, third, and fourth layers, 14, 16, and 18, respectively. FIG. 17 is an enlarged cross-sectional view of the portion of the sweat collection device within circle 17 of FIG. 16. FIG. 17 and FIG. 18 highlight the upper layer 12, the second layer 14, the third layer 16, the fourth layer 18, the lower layer 20, and the adhesive liner 25 (not shown in FIG. 18).

After the assay wells 32 and 40 are formed and the sweat sensing device 10 is assembled, a desired biochemical or chemical assay material, described in detail below, may be disposed therein. The assay wells 32 and 40 may then be closed with an adhesive layer 41, formed from any desired flexible material, such as the same material as the lower layer 20 to which the adhesive layer 41 is attached.

The sweat channels and ports may be formed in the second, third, fourth, and fifth layers 14, 16, 18, and 20 by any desired means, such as with a laser, or die cut.

Referring now to FIG. 1, one embodiment of the improved EEMS system is shown generally at 50, and is comprised of the sweat sensing device 52 having an architecture that is suitable for sweat collection, sweat analysis, and multimodal sensing. The illustrated sweat sensing device 52 of the EEMS system 50 has a first or persistent portion 52A and a second or disposable portion 52B that defines a sweat analysis platform. The persistent portion 52A may include a battery 54, such as a 3.8 V/30 mAh LiPo, rechargeable cell battery that powers the rest of the electronic components, described below. This electrical power may be regulated by an integrated circuit (IC) 56 that feeds appropriate voltages and current to the various processors and sensors onboard the sweat sensing device 52.

A processor 58 performs routine housekeeping, for example, managing the memory, the data transfer, and sensor settings of the device in concert with a smart device (not shown), such as, for example, a smart phone. A power transfer and communications unit 60 allows for power and data to be telemetered to the disposable portion 52B. The persistent portion 52A of the sweat sensing device 52 may include: a bio-impedance sensor 62 for measuring bio-impedance, a bio-potential sensor 64 for measuring bio-potentials, an optical sensor 66 for measuring optical signals, such as pulse, pulse arrival time (PAT), and oxygen saturation (SpO2), and an inertial measurement unit 68 for measuring inertial motion, such as acceleration and angular velocity, and orientation, such as magnetometry.

The disposable portion 52B may include a sweat sensor 70 for measuring sweat rate and sweat composition. The sweat sensing device 52 has the appropriate skin/human interfaces for each applicable sensing modality, including for example electrodes 72 for bio-impedance and bio-potential recording, light-emitting diodes (LEDs) 74 and photodetector diodes (PDs) 76 for optical sensing, and a microfluidic channel 26 and an inlet port (not shown in FIG. 1, but similar to the sweat inlet port 42 of the microfluidic channels 26 shown in FIG. 15, for sweat collection and analysis. The system may optionally include additional sensors, for example, a sensor for detecting fluorescence.

In accordance with some embodiments of the invention, the disposable portion 52B may include a processor 78 as shown in FIG. 1. Any desired processor may be used, however processors 78 with integrated communications and data processing and storage capability are preferred. For example, the Nordic Semiconductor nRF52832 device contains a Cortex-M4F (floating-point) processor with a 512 kB Flash, a 64 kB RAM, and an integrated wireless radio for data processing and telemetering to a smart device (not shown in FIG. 1). The processor 78 is configured to handle changes to each sensor's configuration and settings, enabling different operating modes for the complete system.

In accordance with some embodiments, the processor 78 may facilitate the combined operation of the persistent portion 52A and the disposable portion 52B of the sweat sensing device 52. That is, the disposable portion 52B may contain electronic components with power and data provided by the processor 78 to enable its functionality. In turn, the disposable portion 52B may perform required analysis, process data with its own processor, for example with a RF430RFL152H processor available from Texas Instruments, and telemeter this information back to the host processor 58 on the persistent portion 52A using near-field communications (NFC) or other equivalent communications protocols. It will be understood that the disposable portion 52B may include any of the sensors described herein as being mounted on the persistent portion 52A.

In accordance with some embodiments, the sensors on the sweat sensing device 52 may collect and record various biometrics when coupled to human skin. The bio-impedance sensor 62 and the bio-potential sensor 64, for example a AFE4300 sensor and a ADS1292 sensor from Texas Instruments, respectively, use the integrated electrodes 72 to couple to the skin. In the case of bio-impedance, the electrodes 72 serve as current injection and voltage measurement points to calculate a complex bio-impedance. This bio-impedance can be used to evaluate, for example, skin hydration, sweat conductivity, and stress levels. To facilitate current injection, the electrodes 72 may use Ag or AgCl for skin contact to reduce junction potentials. The electrodes 72 may be used as common inputs between the bio-impedance and bio-potential sensors 62 and 64 in conjunction with an analog switch, for example, complementary metal oxide semiconductor field-effect transistor (MOSFET), a double-pole/double-throw switch, and similar switches, such that only one of the two sensors 62 and 64 may run at any given time. This switching process may be rapid compared to the bio-signals being measured, so that data from both sensors 62 and 64 may be collected at a sufficient rate for analysis. The use of the electrodes 72 in this common configuration optimizes sweat sensing device 52 cost and complexity by reducing the overall layout footprint and fabrication steps.

In accordance with some embodiments, the EEMS system 50 contains the optical sensor 66 that may transmit and receive light reflected or scattered by or transmitted through the body. This sensing modality, when used to measure pulsatile blood flow, is known as photoplethysmography (PPG). PPG allows for the collection of biometric signals relating to pulse-arrival time (PAT), pulse-transit time (PTT), heart rate (HR), and blood oxygen saturation (SpO2). The same modality may be used to analyze various components of sweat using either direct optical or fluorescence detection. This requires a suitable sensor system, for example, the MAX30110 sensor from Maxim Integrated Semiconductor, with LEDs 74 and PDs 76 that operate in the visible, near-infrared, and infrared wavelengths. For example, the LEDs 74 may be red, infrared, or green. The use of this spectrum of wavelengths for the PDs 76, for example between about 540 nm and about 940 nm, may produce outputs that are sensitive to concentrations of various species in sweat, for example, chloride, sodium, glucose, and the like.

The illustrated optical sensor or sensors 66 may be used for ambulatory, cuff-less blood pressure monitoring. The optical sensor 66 may detect pulsatile blood flow with either the red, infrared, or green LEDs 74, and thus may parse out key features of the resulting PPG waveform that correspond to the systolic and diastolic phases of the cardiac cycle. Capturing this data from two or more locations on the body provides up to two or more PPG waveforms that can be time-aligned and compared. The time difference between the waveforms' respective peaks and valleys, commonly referred to as PTT, correlates to a user's absolute blood pressure, systolic and diastolic pressure in millimeters of mercury (mmHg).

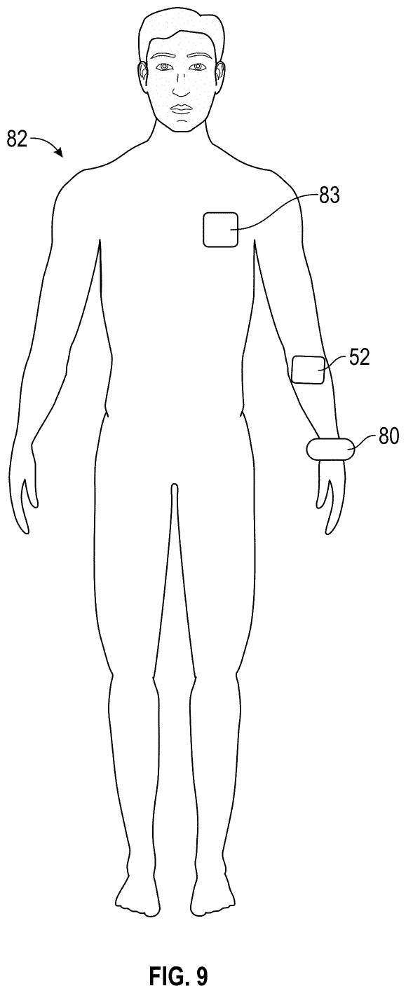

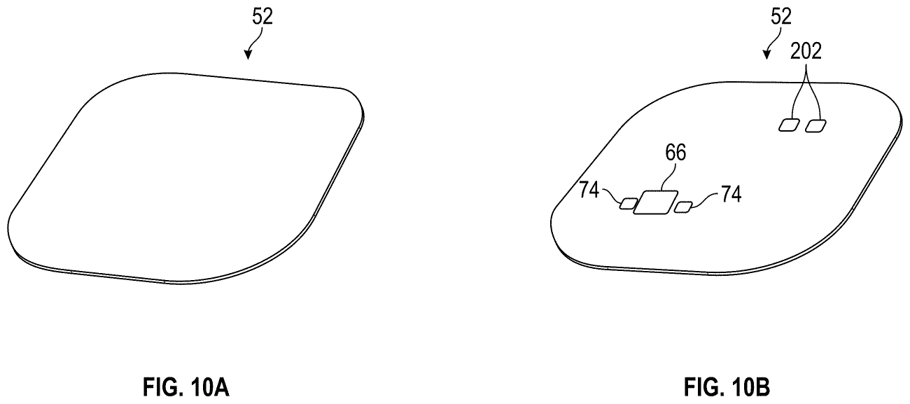

Referring now to FIG. 9, one or more of the sensors is the sweat sensing device 52 of the EEMS system 50, and the other is a smartwatch 80 having a smartwatch application designed to collect PPG waveforms from an integrated optical sensor. Other sensors may be worn by the user. The sweat sensing device 52 measures PPG data that is a time-advanced version of the PPG waveform detected by the smartwatch 80. As shown in FIG. 9, the sweat sensing device 52 may be placed on a person 82 at a location distal to, and on the same body-side, as the wrist-mounted smartwatch 80, for example on the forearm (as shown at 52 in FIG. 9), upper shoulder, or chest (as shown at 83 in FIG. 9), to ensure sufficient time delay between the PPG waveforms. FIG. 10A illustrates first or outwardly facing surface of an exemplary embodiment of the sweat sensing device 52. FIG. 10B illustrates a second or skin-facing surface of the exemplary embodiment of the sweat sensing device 52 shown in FIG. 10A. In FIG. 10B, an optical sensor 66, a plurality of LEDs 74, and a pair of electrodes 202 are shown.

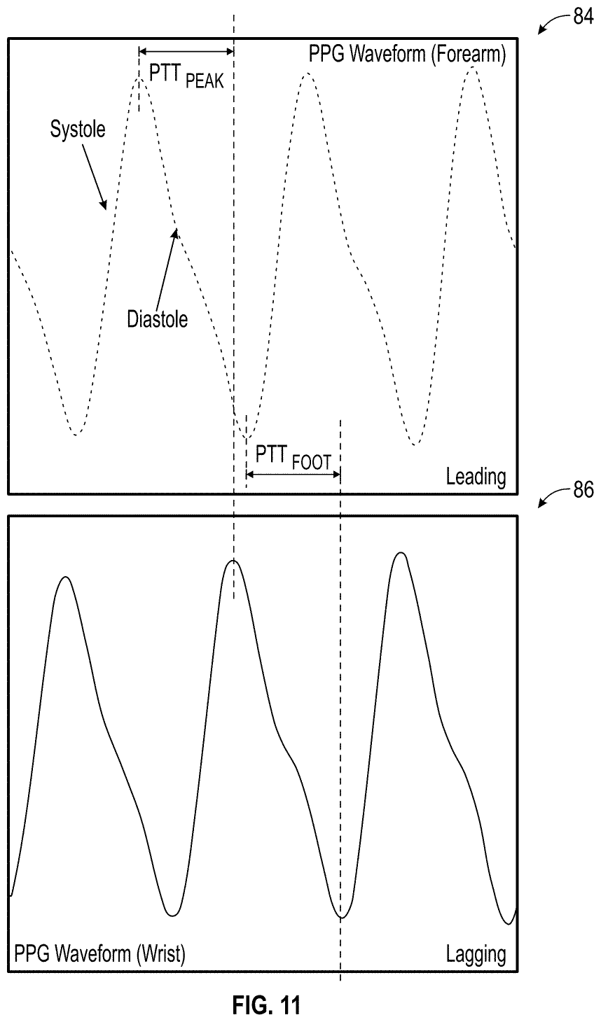

The sweat sensing device 52 sends its PPG data with timestamp information via wireless signal to the smartwatch 80 for processing. The smartwatch 80 aggregates and time aligns the sweat sensing device 52 PPG data (see the graph 84 in FIG. 11) with its own data, i.e., data from the smartwatch 80 (see the graph 86 in FIG. 11), calculating the time difference between the peaks and valleys, PTT_{PEAK} and PPT_{FOOT}, respectively. Through the use of statistical-based and machine-learning models that use the PTT and other physiology metrics, for example gender, height, weight, age, base metabolic rate, and VO2_{MAX} (the velocity at which the maximal oxygen uptake occurs) as predictors to a linear or polynomial regression model, the smartwatch 80 calculates the real-time PTT, pulse wave velocity (PWV), and blood pressure. The smartwatch 80 displays these bio-signals in real-time on the smartwatch display (not shown).

Additionally, the smartwatch 80 may include a pair of electrode leads used to measure ECG data from the wrist of the wearer. The wearer measures ECG signals by placing their finger from the opposing arm in contact with a first one of the electrode leads on the smartwatch. A second one of the electrode leads in the pair is in contact with the wrist underneath the smartwatch 80 to close a loop across the body. In this configuration, the EEMS system 50 measures PPG data from different regions of the body, for example, the forearm, torso, quad muscle, forehead, and other regions) while the smartwatch 80 concurrently measures ECG signals. The smartwatch 80 then aggregates and time aligns PPG data from the EEMS sensors in the sweat sensing device 52 PPG with the smartwatch 80 ECG signal. The smartwatch 80 and/or the EEMS system 50 may calculate the real-time PAT, PWV, and blood pressure signals. The smartwatch 80 then displays the processed real-time PAT, PWV, and blood pressure data on the smartwatch display (not shown).

In the illustrated embodiments, the smartwatch application may also provide a real-time alarm to the user via visual, audible, or haptic signals if any of the calculated PTT, PAT, PVW, or blood pressure exceeds predetermined thresholds. In this case, the smartwatch 80 may make recommendations to the user for corrective courses of actions including, but not limited to, sitting down, pronating, or breathing deeply. Additionally, upon detecting abnormal blood pressure values, the smartwatch application may alter the user's environment or a condition of the environment by playing various sounds, for example, soothing or meditative music, or enabling haptic feedback, for example massaging vibrations, to reduce the user's absolute blood pressure.

The smartwatch application may transmit this information wirelessly to a connected device, for example a personal assistant, a cloud based home control device, and a smart thermostat, that in turn controls the ambient room temperature, plays music in the room, or executes an order for food or pharmaceuticals based on the biometric data collected by the smartwatch 80. For example, the smartwatch 80 may measure high heart rate and blood pressure responses from the EEMS system 50 and smartwatch sensor, and in turn transmit a command to the personal assistant to play soft soothing music in order to reduce stress levels of the wearer.

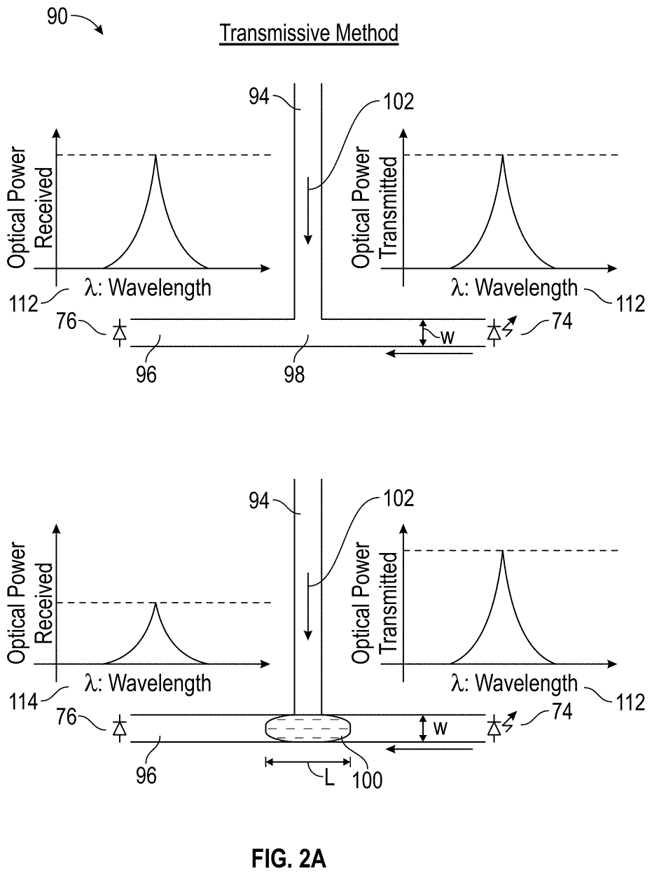

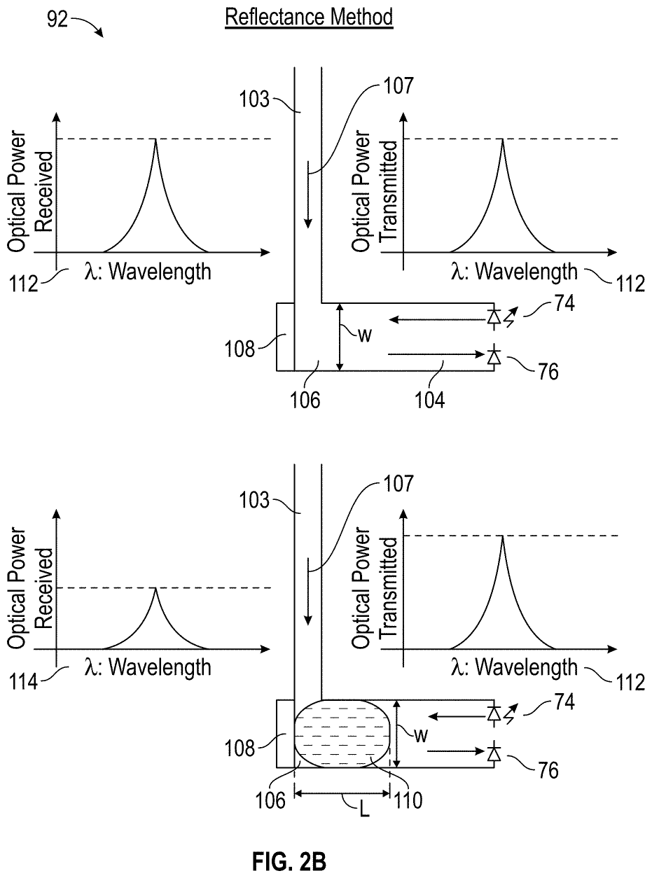

Advantageously, a pair of the LED 74 and the PD 76 may be used to continuously sense a volume of sweat perspired locally on the user. FIG. 2A is a schematic illustration of a first or transmissive method 90 for optical measurement of sweat volume and concentration for use in the wearable EEMS system 50 that uses the attenuation of light as it propagates through a sweat. FIG. 2B is schematic illustration of a second or reflectance method 92 also for optical measurement of sweat volume and concentration for use in the wearable EEMS system 50 that uses the attenuation of light as it propagates through a sweat sample.

FIG. 2A includes a schematic illustration of a first microfluidic channel 94 oriented transversely, and in fluid communication with, a second microfluidic channel 96. A T-junction defines a sweat collection area 98 where the first microfluidic channel 94 is connected to the second microfluidic channel 96. The second microfluidic channel 96 has a width W within the range of about 10 μm to about 1 mm and a known cross-sectional area, such as within the range of about 10 μm to about 500 μm. An LED 74 is mounted at a first end of the second microfluidic channel 96, and a PD 76 is mounted at a second end of the second microfluidic channel 96 in a transmissive mode.

A volume of sweat is propagated from sweat pores on the skin into the first microfluidic channel 94 in the direction of the arrow 102. The volume of sweat will flow until it reaches the collection area 98 where a bead of the sweat 100 forms and has a length L. Because the LED 74 and the PD 76 are configured in the transmissive mode, light illuminated by the LED 74 travels through the bead of sweat 100 and is directly captured by the PD 76. Based on the principles of the Beer-Lambert law for light propagating through a medium, the amount of light absorbed by the bead of sweat 100, and thus the amount of light collected by the PD 76, is directly related to the length L of bead of sweat 100 formed in the collection area 98. By knowing the value of the length L and the cross-sectional area of the second microfluidic channel 96, an overall volume of the bead of sweat 100 may be calculated. This transmissive method 90 mitigates a need for external devices, such as smartphones, cameras, and the like, to optically measure the size of the bead of sweat 100 and allows for local, continuous measurement of sweat volume.

FIG. 2B includes a schematic illustration of a third microfluidic channel 103 oriented transversely, and in fluid communication with, a fourth microfluidic channel 104. A corner junction defines a sweat collection area 106 where the third microfluidic channel 103 is connected to the fourth microfluidic channel 104. The fourth microfluidic channel 104 has the width W within the range of about 10 μm to about 1 mm and a known cross-sectional area, such as within the range of about 10 μm to about 500 μm. An LED 74 and a PD 76 are mounted at a first end of the fourth microfluidic channel 104, and a reflective material defining an optical reflectance plane 108 is mounted at a second end of the fourth microfluidic channel 104 in a reflectance mode. The reflective material may be any desired reflective material such as bright white polyethylene terephthalate (PET), or other similar reflective material.

A volume of sweat is propagated from sweat pores on the skin into the third microfluidic channel 102 in the direction of the arrow 107. The volume of sweat will flow until it reaches the collection area 106 where a bead of the sweat 110 forms and has the length L. Because the LED 74, the PD 76, and the reflectance plane 108 are configured in the reflectance mode, light illuminated by the LED 74 travels through the bead of sweat 110, reflects off of the reflectance plane 108, and is captured by the PD 76. As discussed above, and based on the principles of the Beer-Lambert law for light propagating through a medium, the amount of light absorbed by the bead of sweat 110, and thus the amount of light collected by the PD 76, is directly related to the length L of bead of sweat 110 formed in the collection area 106. By knowing the value of the length L and the cross-sectional area of the fourth microfluidic channel 104, an overall volume of the bead of sweat 110 may be calculated. This reflectance method 92 also mitigates a need for external devices, such as smartphones, cameras, and the like, to optically measure the size of the bead of sweat 110 and allows for local, continuous measurement of sweat volume.

The pair of the LED 74 and the PD 76 shown in FIGS. 2A and 2B may be used to sense a concentration of a specific species in sweat that is collected off the body of the user. The beads of sweat 100, 110 positioned in the collection areas 98 and 106, respectively, will interact with the light propagating from the LED 74 to the PD 76. The interaction of sweat with light may take many forms, including absorption, reflection, refraction, scattering, or fluorescence, each of which may be limited to certain ranges of wavelengths. The strength of this interaction relates to the concentration of certain species interest, for example, chloride, sodium, glucose, and lactate, in the sweat solution. The intensity of light received on the PD 76 thus varies with concentration of the species. Specific species may be targeted based on imaging parameters including the wavelength 112 of light from the LED 74, for example between about 540 nm and about 940 nm, and the wavelength 114 of the light received by the PD 76. These wavelengths 112 and 114 may be controlled through selection of the LED 74, the PD 76, and optical filters (not shown).

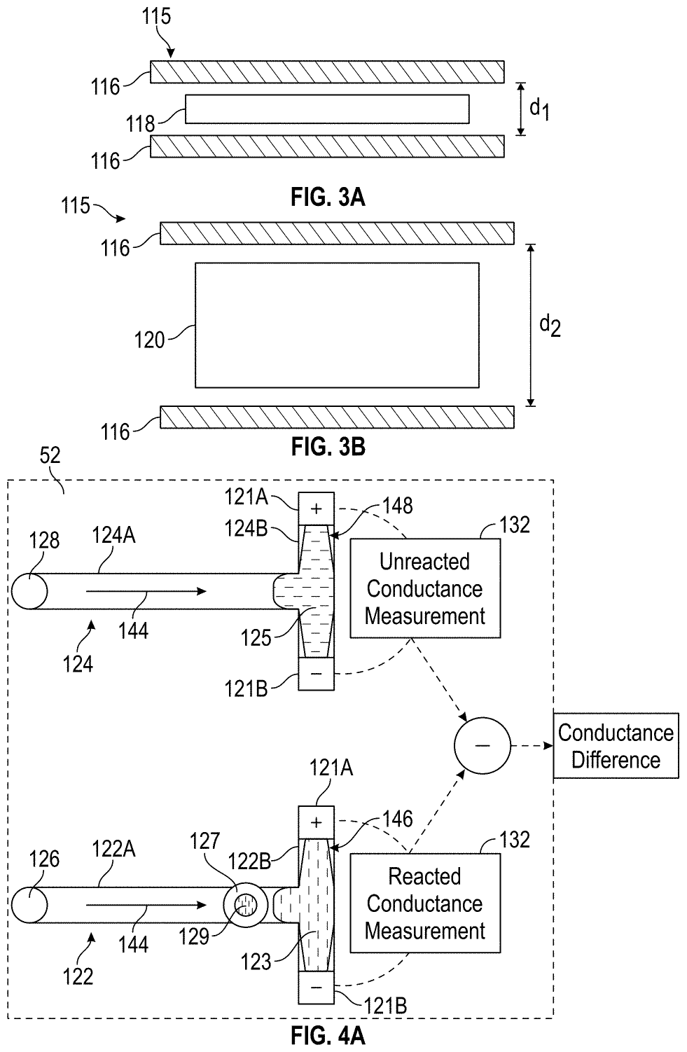

A volume of sweat collected may be calculated using novel structures in a microfluidics platform, such as the sweat sensing device 52. One such structure is a capacitor 115 configured to measure capacitance between two metal plates 116. The metal plates 116 are embedded in the sweat sensing device 52 and spaced apart a first distance d1, as shown in FIGS. 3A and 3B. The distance d1 may be any desired distance, such as a distance between about 10 μm and about 1 mm. The metal plates 116 may be formed from any desired metal, such as gold, silver, or copper metals. A super absorbent polymer (SAP) material 118 is disposed between the metal plates 116 and is in fluid communication with a microfluidic channel in the sweat sensing device 52, such as the sweat channel 26. The SAP material 118 may be any desired SAP material, such as SA60S material manufactured by Sumitomo Seika. Advantageously, the SAP material 118 may expand to a known volume based on the amount of liquid, i.e., sweat in the embodiments of the invention disclosed herein, that the SAP material 118 absorbs.

As the SAP material 118 absorbs sweat, the SAP material expands, as shown at 120 in FIG. 3B, such that the distance between the metal plates 116 increases to a second distance d2, such as a distance between about 10 μm and about 1 mm. This expansion of the SAP material 118 increases the distance between the metal plates 116 and increases the dielectric constant of the sweat separating them, both of which alter the capacitance. By measuring a change in capacitance as the sweat is absorbed into the SAP material 118, an effective volume of sweat absorbed by the SAP material 120 may be calculated. The capacitance may be measured continuously using an Application-Specific Integrated Circuit (ASIC), such as the FDC1004 circuit manufactured by Texas Instruments. Such an ASIC provides an output of absolute capacitance values for most any metal plate geometry.

In the embodiment illustrated in FIGS. 3A and 3B, the metal plates 116 may have any desired shape, such as rectangular, square, circular, and other geometric shapes having a known area or an area that can be calculated. The use of the ASIC further allows for easy integration into the sweat sensing device 52 without the need for external equipment or a measurement device, such as an LCR meter. The ASIC in the illustrated embodiment is capable of measuring capacitance values within the range of about 0 pF to about 100 pF. Thus, the capability of the ASIC provides design guidelines for the first and second distances d1 and d2 between the metal plates 116 and the choice of SAP material 118.

Capacitance may also be calculated in a conventional manner, such as with the formulas in Table 1.

TABLE 1

| | |
|---|---|
| ${C}_{BEFORE} = \frac{{ɛ}_{GEL} * A}{d 1}$ | ${C}_{AFTER} = \frac{( {ɛ}_{GEL} \vert \vert {ɛ}_{SWEAT} ) * A}{d 2}$ |
| wherein: | wherein: |
| C_{BEFORE} is capacitance of the SAP | C_{AFTER} is capacitance of the SAP |
| material 118. | material 120. |
| A is the area of the plates 116. | A is the area of the plates 116. |
| ε_{GEL} is the permittivity of the SAP | ε_{GEL} ∥ ε_{SWEAT} is the permittivity of |
| material 118. | the SAP material 120. |

Alternatively, the SAP material 118 may be placed electrically outside of the capacitor 115. For example, the SAP material 118 may be placed in a ring around the metal plates 116, but mechanically connected to the metal plates 116 such that swelling of the SAP material 118 changes the distance between the metal plates 116, for example from the distance d1 to the distance d2, but does not alter the dielectric constant of the sweat separating metal plates 116. Additionally, the SAP material 118 may be placed between one metal plate 116 and a rigid surface (not shown) so that the metal plates 116 are pushed closer together by swelling of the SAP material 120.

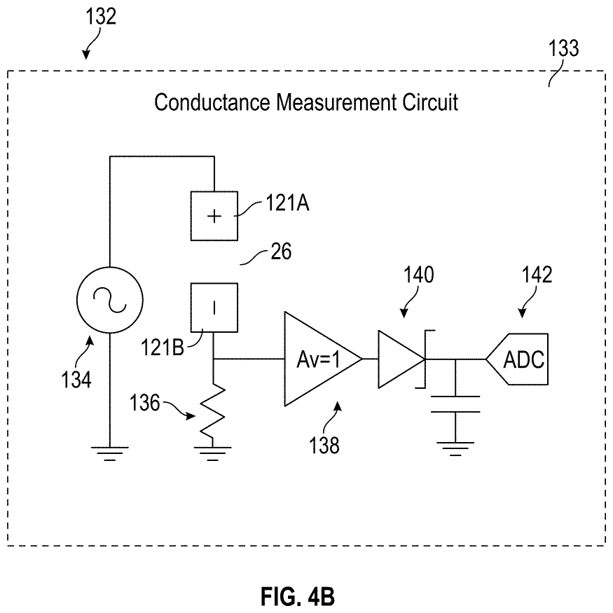

In another embodiment of the invention, a concentration of specific species in the sweat solution may be measured via a differential measurement method, best shown in FIGS. 4A and 4B. FIG. 4A is a schematic illustration of one embodiment of a portion of the sweat sensing device 52 of the wearable EEMS system 50 including a first sweat collection channel 122 with an analyte well and a second sweat collection channel 124 without an analyte well.

The first sweat collection channel 122 includes a first microfluidic channel 122A having a first end defining a first sweat inlet port 126 and a second end. The second end of the first microfluidic channel 122A is oriented transversely to, and is in fluid communication with, a second microfluidic channel 122B. A T-junction defines a sweat collection area 123 where the first microfluidic channel 122A is connected to the second microfluidic channel 122B. Positive and negative electrical terminals, 121A and 121B, respectively, are attached at opposite ends of the microfluidic channel 122B. An analyte well 127 is formed in the second microfluidic channel 122B near the sweat collection area 123. A conductance measurement circuit 132 (described below) is electrically connected to the positive and negative electrical terminals 121A, 121B at the opposite ends of the second microfluidic channel 122B.

The second sweat collection channel 124 is similar to the first sweat collection channel but does not include the analyte well. The second sweat collection channel 124 includes a first microfluidic channel 124A having a first end defining a second sweat inlet port 128 and a second end. The second end of the first microfluidic channel 124A is oriented transversely to, and is in fluid communication with, a second microfluidic channel 124B. A T-junction defines a sweat collection area 125 where the first microfluidic channel 124A is connected to the second microfluidic channel 124B. The positive and negative electrical terminals, 121A and 121B, respectively, are attached at opposite ends of the second microfluidic channel 124B. The conductance measurement circuit 132 (described below) is electrically connected to the positive and negative electrical terminals 121A, 121B at the opposite ends of the second microfluidic channel 124B.

The conductance measurement circuit 132, shown in FIG. 4B, may be configured as a microprocessor 133, such as the RF430RFL152H microprocessor manufactured by Texas Instruments. The microprocessor 133 may measure electrical conductivity, and may include a source of AC voltage 134, a reference resistor 136, unity gain buffer 138, such as the TSV632 unity gain buffer manufactured by ST Microelectronics, a Schottky diode 140, for example an SMS7640-040LF diode manufactured by Skyworks Solutions, Inc., and a data converter 142, such as an analog-to-digital converter.

The two sweat collection channels 122 and 124 are co-located in the sweat sensing device 52 and collect sweat in a similar manner. In the first sweat collection channel 122, sweat enters the first microfluidic channel 122A through the first sweat inlet port 126 and travels in the direction of the arrow 144. As a volume of sweat 146 moves toward the sweat collection area 123, the sweat flows through the analyte well 127. A chemical assay 129 in the analyte well 127 reacts with a specific analyte species in the sweat, for example, chloride, sodium, glucose, and lactate, in a way that changes the electrical mobility of charge carriers in the sweat, and thus changes the electrical conductivity of the sweat. The electrical conductivity within the sweat collection channel 122 may be measured with the conductive measurement circuit 132.

Similarly, sweat enters the first microfluidic channel 124A of the second sweat collection channel 124 through the second sweat inlet port 128 and travels in the direction of the arrow 144, and defines a volume of sweat 148 that moves toward the sweat collection area 125. The electrical conductivity within the sweat collection channel 124 may be measured with the conductive measurement circuit 132.

By measuring the electrical conductivity in both the first sweat collection channel 122 and the second sweat collection channel 124, and calculating a conductive difference, the net change in conductivity will vary monotonically with the amount of analyte that reacts with the chemical assay 129. For example, silver chloranilate will react with chloride in the sweat to form a silver chloride precipitate and release chloranilate ions into solution. Since chloranilate is significantly larger than chloride, its electrical mobility is much lower. This reaction thus reduces the electrical conductivity of the sweat. In another example, glucose oxidase catalyzes the oxidation of glucose to produce hydrogen peroxide and D-glucono-1,5-lactone, which hydrolyzes to gluconic acid. At neutral pH, gluconic acid dissociates into H+ and gluconate, both of which increase the electrical conductivity of the sweat solution.

The source of AC voltage 134 drives the voltage divider between the each of the first sweat collection channel 122 and the second sweat collection channel 124 and the reference resistor 136. This divider produces an attenuated version of the source of AC voltage whose amplitude depends on the conductivity of the sweat solution. This amplitude, buffered by the unity-gain buffer 138, is detected with the Schottky diode 140 and the data converter 142. The detected amplitude may be used to calculate the original impedance of the sweat collection channels 122 and 124, thus also the conductivity.

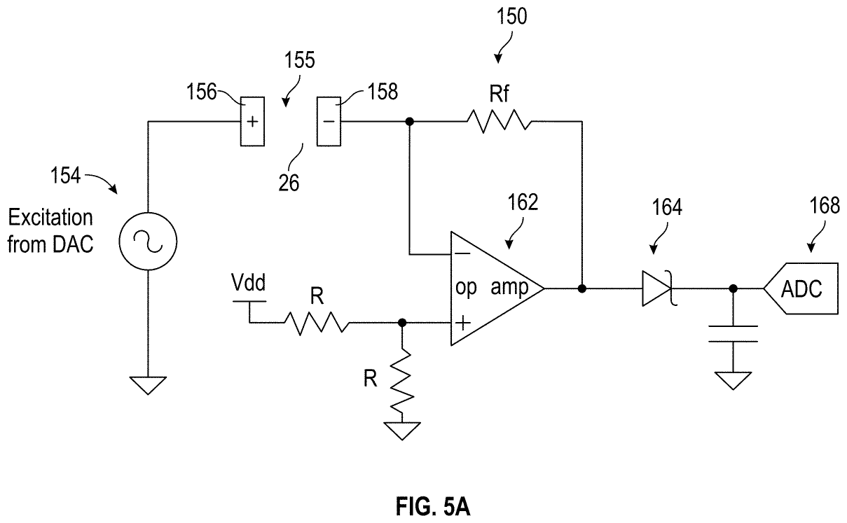

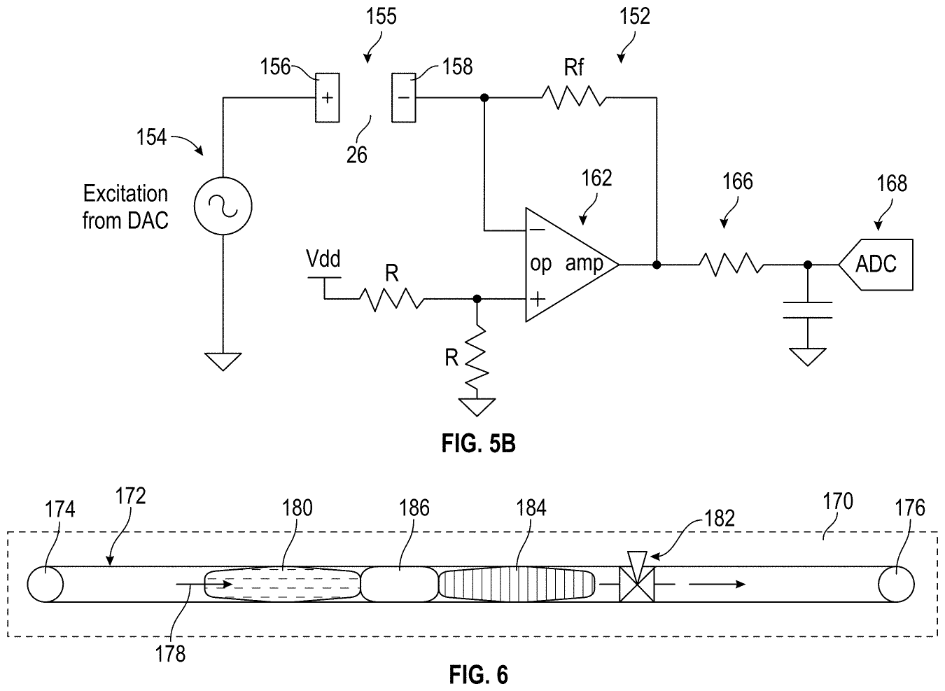

Additional embodiments of the invention are illustrated in FIGS. 5A and 5B. For example, FIG. 5A is a diagram illustrating a first embodiment 150 of a method of measuring impedance of a sweat channel in the wearable sweat sensing device 52 of the EEMS system 50, showing amplifier output being directed to a diode detector circuit.

FIG. 5B is a diagram illustrating a second embodiment 152 of a method of measuring impedance of a sweat channel in the wearable sweat sensing device 52 of the EEMS system 50, showing amplifier output being directed to an anti-aliasing filter.

As shown in the embodiments illustrated in FIGS. 5A and 5b, the impedance of the sweat channel 26 may be measured using an inverting amplifier topology. For example, a digital to analog converter (DAC) 154 generates a sinusoidal signal and drives a positive plate 156 of a capacitor 155 in the sweat channel 26, as shown in FIGS. 5A and 5B. A negative plate 158 is held at mid-scale. The sinusoid signal generated by the DAC 154 is symmetrical around the mid-scale voltage, ensuring that the plates 156 and 158 in the sweat channel 26 do not build up a DC offset. The gain of an amplifier 162 is set by adjusting the feedback resistor Rf. An output of the amplifier 162 is fed into either a diode detector circuit 164, as shown in FIG. 5A, or into an anti-aliasing filter 166, as shown in FIG. 5B, before the sinusoid signal is sent to the analog to digital converter ADC 168.

As shown in FIG. 5A, the sinusoid signal is rectified by the diode in the diode detector circuit 164, and filtered by the capacitor 155 to generate a DC scalar magnitude that encompasses the real and imaginary components. Alternatively, if faster ADCs 168, DACs 154 and a more powerful microcontroller (not shown in FIGS. 5A and 5B) are available, such as the KL02 microcontroller manufactured by Freescale Semiconductor, digital synchronous demodulation techniques and/or phase computations may be made taking advantage of real and imaginary values. Additionally, the sinusoid frequency may be swept and the real and imaginary components computed for multiple sweat channels and the results may be compared in the frequency domain by performing a digital fast Fourier transform (FFT) algorithm.

In one alternate embodiment, a first pair of electrodes, such as the electrodes 156 and 158, is used to drive a sinusoidal current of known amplitude through the sweat channel 26, while a second pair of electrodes 156, 158 is used to measure the resulting voltage. This four-electrode approach reduces the impact of junction potentials that arise at the electrodes 156, 158 when passing electrical current into fluids, such as sweat.

Referring now to FIG. 6, a portion of one embodiment of the sweat sensing device 52 of the EEMS system 50 is shown positioned on the skin 170 of a user. FIG. 6 illustrates a microfluidic channel 172 having an inlet port 174 and an active pharmaceutical agent (APA) outlet port 176, such that sweat 180 flows through the microfluidic channel 172 in the direction of the arrow 178. The illustrated microfluidic channel 172 further includes a microvalve 182, which may be controlled actively, i.e., electronically, or passively. A volume of an active APA 184 is introduced into the microfluidic channel 172 and positioned at location between the microvalve 182 and the inlet port 174 from a source of APA (not shown). An air bubble 186 is also introduced into the microfluidic channel 172 and positioned at location adjacent the APA 184, between the inlet port 174 and the APA 184 from a source of air (not shown). The EEMS system 50 may include microprocessor (not shown) to actuate the microvalve 182. Any desired microprocessor may be used, such as the nRF52832 microprocessor manufactured by Nordic Semiconductor, or the RF430RFL152H microprocessor manufactured by Texas Instruments.

Advantageously, the microfluidic channel 172 illustrated in FIG. 6 is configured to collect sweat 180 from the user, measure electrical signals, and deliver pharmaceutical drugs to the user.

Specifically, in operation, fluid pressure is exerted by the volume of sweat 180 that flows through the sweat inlet port 174 into the microfluidic channel 172 on the air bubble 186. For example, as the sweat 180 propagates through the microfluidic channel 172, it may exert a pressure on the air bubble 186 with the range of about 0.25 kPa to about 6 kPa, causing air bubble to move in the direction of the arrow 178. The pressure exerted on the air bubble 186 will urge the air bubble 186 against the APA 184, thus urging the APA 184 to an inlet of the microvalve 182.

Upon entering the microfluidic channel 172, the sweat 180 may be analyzed to determine volume and/or species concentration using any of the methods described herein. Once the sweat 18 in the microfluidic channel 172 is analyzed, the microprocessor (not shown) may then actuate the microvalve 182. The analysis of the sweat 180 is critical because the microprocessor (not shown) may be configured to determine the volume of APA 184 to dispense onto the skin of the user via the APA outlet 176, depending on the sensed and measured characteristics of the collected sweat 178.

For example, the APA 184 may be a topical form of insulin that is dispensed in a precise amount via an electronically-controlled microvalve 182 based on an amount, i.e., a volume and concentration, of glucose in the analyzed sweat 180. The additional advantage of this embodiment is that it mitigates the need for a microfluidic pump to dispense the APA 184, relying instead on the fluid pressure inherently exerted by the sweat 180 as it propagates from the skin into the sweat inlet 174.

In one embodiment, a cross-sectional area of the portion of the microfluidic channel 172 containing the APA 184 may have a larger cross-sectional area than a remainder of the microfluidic channel 172. The larger relative size of the portion of the microfluidic channel 172 containing the APA 184 increases the force applied to the APA 184 at a given pressure, for example to displace a highly viscous APA 184. Because the linear displacement of sweat 180 and the APA 184 are identical at a constant air bubble 186 pressure, variations in cross-sectional area in the microfluidic channel 172 can also be used in conjunction with the microvalve 182 to control the volumetric rate at which APA 184 is dispensed.

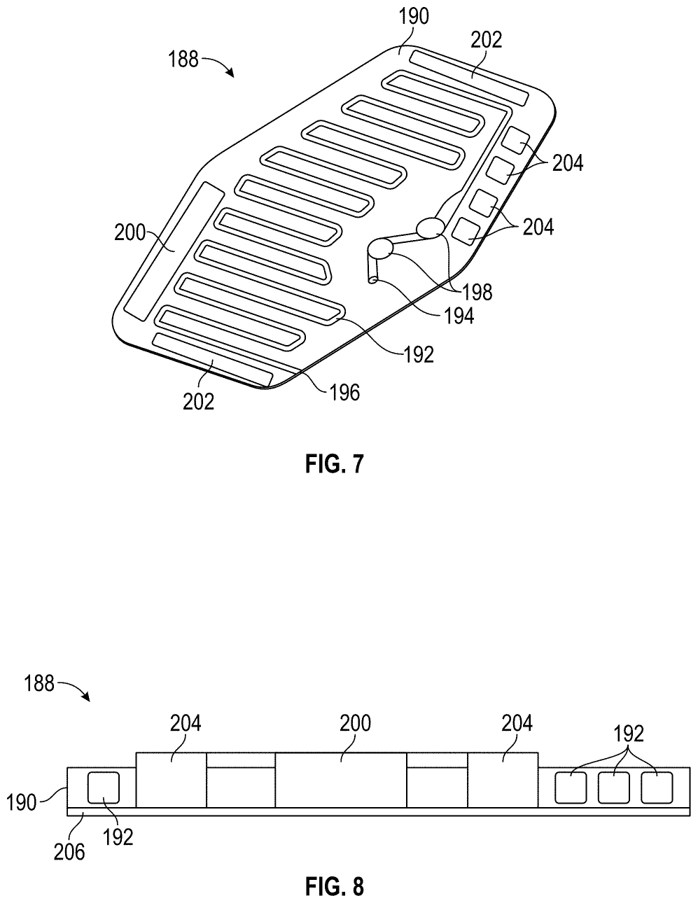

Referring now to FIG. 7, an additional embodiment of the improved sweat collection device with integrated biosensors and microfluidics, and for use with the improved wearable EEMS system 50, is shown at 188. The sweat collection device 188 is similar to the sweat sensing device 10 and includes a substantially flexible body 190 comprised of one or more layers. A lower or skin-facing surface may include a removable adhesive liner 206 formed from any desired flexible and air/oxygen impermeable material.

One or more microfluidic or sweat channels 192 are formed in the body 190. The sweat channel 192 includes a sweat inlet port 194 at a first end thereof and a sweat outlet end 196 at a peripheral edge of the sweat sensing device 188 and positioned to allow sweat to exit the sweat channel 192. The sweat channel 192 may also include a one or more biochemical or assay wells 198 near the sweat inlet port 194. The assay wells 198 contains colorimetric assays for visual analysis of sweat chemistry.

The sweat sensing device 188 may also include electrodes 202, for example for use with one or more of an ECG, an EMG, or an EEG. The sweat sensing device 188 may also include sensors 204 embedded in the one or more layers of the body 190. The sensors 204 may include, but are not limited to an optical sensor configured to measure pulse rate, heart rate, respiration rate, and the like, a temperature sensor, such as a thermistor or a thermocouple, an accelerometer for motion and motion artifact sensing, a bioimpedance sensor, an IMU sensor, and an ECG sensor. These sensors 204 have associated electronic components, such as a microprocessor, a battery, memory, power regulation means, communications means, and energy harvest means, also embedded in the one or more layers of the body 190 adjacent sweat channel 192, as shown at 200.

Referring now to FIG. 8, a schematic, cross-sectional view of the sweat sensing device 188 shows the individual electronics components within the body 190. The body 190 has one or more sweat channels 192, the electronic components 200, and two or more sensors 204. The sweat sensing device 188 may include any desired number and combination of the sensors 204, such as an ECG sensor and an optical sensor, an ECG sensor and a bioimpedance sensor, two optical sensors, and other combinations of the sensors 204.

A surface of the body 190 may be treated with acrylic or silicone based adhesives to provide sufficient adhesion for the electronic components 200 and the sensors 204 embedded in the opening slots formed in the body 190. A lower or skin-facing surface of the body may be transparent and configured to provide a moisture barrier and an optical path for optical sensing of pulse waveform and pulse oximetry signal during sweat collection.

Advantageously, the sensing device 188 may be configured to use its embedded electronic components 200 and sensors 204 to measure heart rate, heart rate variability, pulse, pulse arrival time, pulse transit time, pulse wave velocity, respiration rate, respiration rate variability, skin temperature, core body temperature, blood pressure, gait, posture, muscle potential, motion, bioimpedance, stride length muscle activity, respiration rate, motion, and/or motion artifacts during an exercise. The sweat channels 192 simultaneously collect sweat during exercise and measure sweat composition, including levels of sweat metabolite, electrolyte, toxins, instantaneous sweat rate, and average sweat rate in the sweat channels 192. Additionally, the electronic components 200 and sensors 204 may be configured to measure EMG, motion and motion artifacts during exercise. The sweat channels 192 simultaneously collect sweat during exercise and measure lactate and glucose levels in sweat.

The assay wells 198 of the sweat channels 192 may contain colorimetric assay material, for example, chloride assay and glucose assay, that react and create a color change upon contact with sweat flowing through the sweat channels 192.

To use the sweat collection device 52, the removable adhesive liner 206 is removed from the skin-facing surface, and the skin-facing surface is affixed to the skin of the person being monitored. The sweat collection device 52 may be adhered anywhere on the person being monitored, including but not limited to the forearm, head, shoulders, arms, hands, torso, chest, legs and feet. Additionally, the sweat collection device 52 may collect within the range of about 5 μL to about 500 μL of sweat from the person being monitored during, after, or both during and after completion of an exercise or physical exertion routine or through electrochemical sweat induction by application of, for example, pilocarpine and electrical stimulation.

The principle and mode of operation of this invention have been explained and illustrated in its preferred embodiment. However, it must be understood that this invention may be practiced otherwise than as specifically explained and illustrated without departing from its spirit or scope.

What is claimed is:

1. A system for collecting and analyzing sweat comprising:
a sweat sensing device having at least one sensor configured to sense one of physiologic information, biologic information, biochemical information, and biometric information, from skin of a person to whom the sweat sensing device is attached;
a wireless transmitter mounted to the sweat sensing device and electrically connected to the at least one sensor;
an interactive console configured to produce a real time feedback to an operator of the system for collecting and analyzing sweat;
wherein the interactive console includes a wireless receiver and is in wireless communication with the sweat sensing device; and
wherein the interactive console is further configured to receive data from the at least one sensor, display the data received from the at least one sensor, adjust an environmental condition based on the sensor data received, and display the adjustment to the environmental condition.

2. The system according to claim 1, wherein the at least one sensor is removably attached to the sweat sensing device, and is removable from the sweat sensing device upon completion of use.

3. The system according to claim 1, wherein the one of physiologic information, biologic information, biochemical information, and biometric information is selected from the group consisting of sweat composition, sweat rate, heart rate, heart rate variability, pulse, pulse arrival time, pulse transit time, pulse wave velocity, respiration rate, respiration rate variability, skin temperature, core temperature, blood pressure, gait, posture, muscle potential, motion, bioimpedance, and stride length.

4. The system according to claim 3, wherein the system for collecting and analyzing sweat is a wearable electronics-enabled microfluidics sensor (EEMS) system.

5. The system according to claim 1, wherein the sweat sensing device includes a persistent portion and a disposable portion removably attached to the persistent portion.

6. The system according to claim 5, wherein the disposable portion defines a sweat analysis platform.

7. The system according to claim 6, wherein the disposable portion further includes the at least one sensor, and wherein the disposable portion is removable upon completion of use.

8. The system according to claim 5, wherein the persistent portion includes plurality of sensors embedded in the persistent portion and configured to sense one or more of the physiologic information, biologic information, biochemical information, and biometric information.

9. The system according to claim 8, wherein the persistent portion further includes at least one of a microprocessor, a battery, memory, power regulation means, communications means, and energy harvest means embedded in the persistent portion and electrically connected to the plurality of sensors.

10. The system according to claim 1, wherein the at least one sensor is an electronic sensor that measures one of heart rate and body temperature and wherein the sweat sensing device simultaneously captures sweat in a microfluidic channel formed therein.

11. The system according to claim 1, wherein the at least one sensor is an electronic sensor that measures one of heart rate and body temperature and wherein the sweat sensing device simultaneously measures one of instantaneous sweat rate and average sweat rate in sweat captured in a microfluidic channel formed in the sweat sensing device.

12. The system according to claim 1, wherein the at least one sensor is an electronic sensor that measures one of heart rate and body temperature and wherein the sweat sensing device simultaneously measures one of electrolyte, metabolite, and toxins in sweat captured in a microfluidic channel formed in the sweat sensing device.

13. The system according to claim 1, wherein the at least one sensor is an electronic sensor that measures one of heart rate and motion and wherein the sweat sensing device simultaneously measures one of instantaneous sweat rate and average sweat rate in sweat captured in a microfluidic channel formed in the sweat sensing device.

14. The system according to claim 1, wherein the at least one sensor is an electronic sensor that measures one of heart rate and motion and wherein the sweat sensing device simultaneously measures one of electrolyte, metabolite, and toxins in sweat captured in a microfluidic channel formed in the sweat sensing device.

15. The system according to claim 1, wherein the at least one sensor is an electronic sensor that measures one of electromyography and motion and wherein the sweat sensing device simultaneously measures one of glucose concentration and lactate concentration in sweat captured in a microfluidic channel formed in the sweat sensing device.

16. The system according to claim 1, wherein the at least one sensor is an electronic sensor that is embedded within the sweat sensing device adjacent to a microfluidic channel formed in the sweat sensing device.

17. The system according to claim 1, wherein the interactive console is connected to a cloud server and transmits the data received from the at least one sensor to the cloud server.

18. A system for collecting and analyzing sweat comprising:
a sweat sensing device including a persistent portion and a disposable portion removably attached to the persistent portion;
wherein the disposable portion defines a sweat analysis platform;
wherein the disposable portion is removable upon completion of use; and
wherein the persistent portion includes a plurality of sensors embedded therein and configured to sense one of physiologic information, biologic information, biochemical information, and biometric information, from skin of a person to whom the sweat sensing device is attached;
a wireless transmitter mounted to the sweat sensing device and electrically connected to the at least one sensor;
an interactive console configured to produce a real time feedback to an operator of the system for collecting and analyzing sweat;
wherein the interactive console includes a wireless receiver and is in wireless communication with the sweat sensing device; and
wherein the interactive console is further configured to receive data from the at least one sensor, display the data received from the at least one sensor, adjust an environmental condition based on the sensor data received, and display the adjustment to the environmental condition.

19. The system according to claim 18, wherein the persistent portion further includes at least one of a microprocessor, a battery, memory, power regulation means, communications means, and energy harvest means embedded in the persistent portion and electrically connected to the plurality of sensors.

20. The system according to claim 19, wherein the plurality of sensors include at least an electronic sensor that measures one of heart rate and body temperature and wherein the sweat sensing device simultaneously captures sweat in a microfluidic channel formed in the persistent portion therein;
wherein the sweat sensing device simultaneously measures one of instantaneous sweat rate, average sweat rate, electrolyte, metabolite, and toxins in sweat captured in a microfluidic channel formed in the sweat sensing device.
